(19) United States
(12) Patent Application Publication
(10) Pub. No.: US 2014/0213908 A1
(43) Pub. Date: Jul. 31, 2014

(54) PORTABLE ELECTRONIC DEVICE HAVING HEART RATE MONITORING FUNCTION

(71) Applicant: HON HAI PRECISION INDUSTRY CO., LTD., New Taipei (TW)

(72) Inventor: CHIH-CHUNG WENG, New Taipei (TW)

(73) Assignee: HON HAI PRECISION INDUSTRY CO., LTD., New Taipei (TW)

(21) Appl. No.: 13/861,395

(22) Filed: Apr. 12, 2013

(30) Foreign Application Priority Data
Jan. 29, 2013 (TW) 102103347

(51) Int. Cl. A61B 5/024; A61B 5/00

(52) U.S. Cl. CPC A61B 5/02444; A61B 5/0059
USPC 600/475; 600/473

(57) ABSTRACT

A portable electronic device includes an infrared touch screen and a processor. The infrared touch screen includes a detection unit, a filter unit, and a signal conversion unit. The detection unit detects the intensity of the infrared beams reflected by a user's body part placed on the infrared touch screen, and converts the detected intensity of the infrared beam to electrical signals. The filter unit filters the electrical signals and amplifies the filtered electrical signals. The signal conversion unit converts the electrical signals to digital signals. The processor includes an information processing module and a display control module. The information processing module processes the digital signals to obtain the heart rate information of the user, and creates a heart rate monitoring result according to the heart rate information of the user. The display control module controls the infrared touch screen to display the heart rate monitoring result.

### BACKGROUND

1. Technical Field

The present disclosure relates to portable electronic devices, and particularly to, a portable electronic device having a heart rate monitoring function.

2. Description of Related Art

People are concerned about heart disease. However, most of the products for monitoring human hearts and/or pulse are bulky and are inconvenient to carry. Some portable products can be worn on wrist or waist but become a burden to be carried. On the other hand, users commonly carry portable electronic devices having infrared touch panels such as mobile phones, tablet computers, personal digital assistants.

Therefore, what is needed is a portable electronic device having a heart rate monitoring function to alleviate the limitations described above.

### BRIEF DESCRIPTION OF THE DRAWINGS

The components in the drawings are not necessarily drawn to scale, the emphasis instead being placed upon clearly illustrating the principles of the present disclosure. Moreover, in the drawings, like reference numerals designate corresponding sections throughout the several views.

FIG. 1 is a block diagram of a portable electronic device in accordance with an exemplary embodiment.

FIG. 2 is a schematic view of the portable electronic device of FIG. 1 in use, in accordance with the exemplary embodiment.

### DETAILED DESCRIPTION

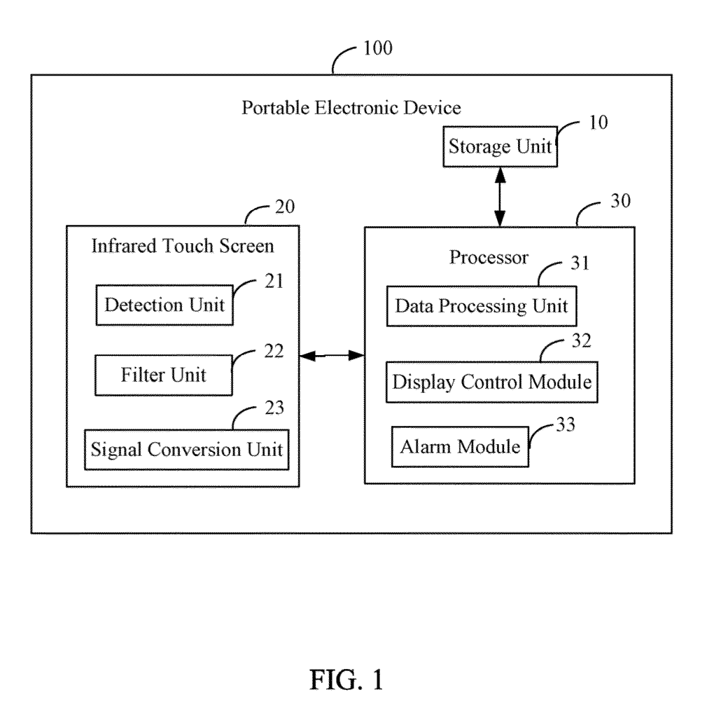

FIG. 1 is an exemplary embodiment of a disclosure of a portable electronic device 100. The portable electronic device 100 can monitor the real-time heart rate of a user via an infrared touch screen. The electronic device 100 is a mobile terminal with a touch screen, such as a mobile phone. In alternative embodiments, the electronic device 100 can be other electronic devices with touch screens, such as an electronic reader, a tablet computer, a digital phone frame or the like.

The portable electronic device 100 includes a storage unit 10, an infrared touch screen 20, and a processor 30. The storage unit 10 stores a reference value of a human heart rate and a reference value range of the human heart rate.

The touch screen 20 detects the pulse of a user whose body part is placed on the infrared touch screen 20, converts the detected pulse data to electrical signals, and transmits the electrical signals to the processor 30. In the embodiment, the touch screen 20 includes a detection unit 21, a filter unit 22, and a signal conversion unit 23.

The processor 30 processes the electrical signals transmitted from the touch screen 20, and controls to display a heart rate figure associated to the processed electrical signals on the infrared touch screen 20. In the embodiment, the processor 30 includes a data processing module 31, a display control module 32, and an alarm module 33.

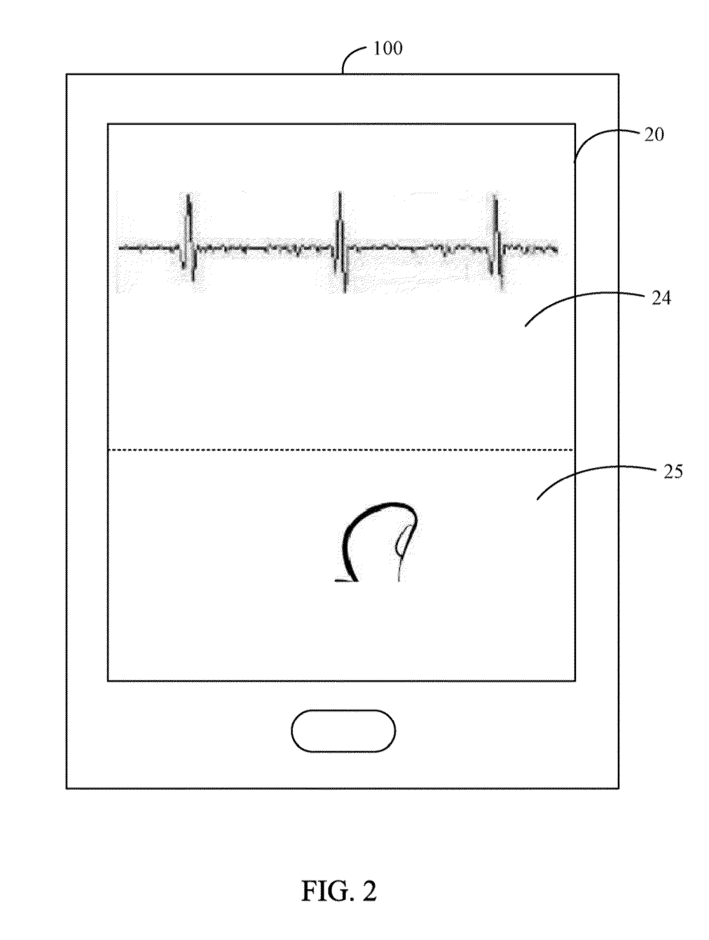

FIG. 2 shows a schematic view of the portable electronic device 100. In the embodiment, when the heart rate monitoring function of the portable electronic device 100 is activated, the display control module 32 controls the infrared touch screen 20 to display a result displaying region 24 and a monitoring region 25. The monitoring region 25 is used for allowing the user to place one part of his/her body thereon for heart rate detection. The result displaying region 24 is used for displaying the results of heart rate monitoring, such as the heart rate figures, information of heart beat or pulse, for example. In an alternative embodiment, the result displaying region 24 is also used for displaying alarm information according to the control signals transmitted from the alarm module 33. The alarm information can be abnormal data detected by the detection unit 21.

In the embodiment, the result displaying region 24 is arranged at the upper potion of the infrared touch screen 20. The monitoring region 25 is arranged at the lower portion of the infrared touch screen 20. In other embodiments, the locations and the sizes of the result displaying region 24 and the monitoring region 25 can be varied according to need.

In the embodiment, when a user places his/her body part (eg. a finger) on the monitoring region 25, the detection unit 21 automatically detects the intensity of the infrared beams reflected by the body part. The detection unit 21 also converts the detected data—the intensity of the reflected infrared beams to electrical signals, and transmits the electrical signals to the filter unit 22. When the heart of the user beats, a transparency of the part of the user decreases, and the brightness of the infrared light reflected by the part of the user increases. When the heart muscle relaxes between beats, a transparency of the part of the user increases, and the brightness of the infrared light reflected by the part of the user decreases. In the embodiment, the part of the body is a finger such as the thumb, or the index finger. In other embodiments, the part of the body is selected from the group consisting of one or more finger, the palm, the wrist, or the earlobe.

The filter unit 22 filters the noises in the electrical signals converted by the detection unit 21, magnifies the filtered electrical signals, and transmits the magnified electrical signals to the signal conversion unit 23. The signal conversion unit 23 converts the magnified electrical signals to digital signals, and transmits the digital signals to the processor 30.

The data processing module 31 of the processor 30 processes the digital signals transmitted from the signal conversion unit 23. In the embodiment, the data processing module 31 shapes and calculates the received digital data to obtain heart rate data of the user. The data processing module 31 also creates a heart rate monitoring result according to the heart rate data of the user. In the embodiment, the heart rate monitoring result can be a heart rate figure or the information of heart beat or pulse. The display control module 32 controls the infrared touch screen 20 to display the heart rate monitoring result of the user on the result displaying region 24. The information of heart beat or pulse is the number of heart beats per minute.

In the embodiment, the data processing module 31 also compares the heart rate data of the user with the reference value of the human heart rate and the value range of the human heart rate stored in the storage unit 10, sends a control signal to the alarm module 33 if the heart rate of the user is not equal to the reference value or beyond the value range of the human heart rate. The alarm module 33 controls the infrared touch screen 20 to display alarm information indicating an unhealthy situation on the result displaying region 24 when the control signal is received. The reference value of the human heart rate and the value range of the human heart rate stored in the storage unit 10 can be varied according to different users.

With such a configuration, when the heart rate monitoring function of the portable electronic device 100 is activated; if the user places a part of his/her body on the infrared touch screen 20 of the portable electronic device 100. The portable electronic device 100 then detects the heart rate data and displays the detected heart rate information on the infrared touch screen 20. The portable electronic device 100 also displays warning information to the user when the heart rate of the user is higher or lower than the reference value or beyond the value range of the human heart rate. The user normally carries a portable electronic device 100 having the infrared touch screen 20, and can monitor the real-time heart rate of the user by using the infrared touch screen 20, which satisfies the demands of the user.

Although the present disclosure has been specifically described on the basis of the embodiments thereof, the disclosure is not to be construed as being limited thereto. Various changes or modifications may be made to the embodiments without departing from the scope and spirit of the disclosure.

What is claimed is:

1. A portable electronic device for monitoring a heart rate of a user, comprising:
an infrared touch screen comprising:
a detection unit, configured to detect intensity of infrared beams reflected by the user's body part placed on the infrared touch screen, and to convert the intensity of the reflected infrared beams to electrical signals;
a filter unit, configured to filter noises in the electrical signals, and to magnify the filtered electrical signals; and
a signal conversion unit, configured to convert the magnified electrical signals to digital signals; and
a processor comprising:
a data processing module, configured to process the digital signals to obtain heart rate data of the user, and to create a heart rate monitoring result according to the heart rate data of the user; and
a display control module, configured to control the infrared touch screen to display the created heart rate monitoring result.

2. The portable electronic device as described in claim 1, further comprising a storage unit storing a reference value of a human heart rate, wherein the processor further comprises an alarm module, the data processing module is further configured to compare the heart rate data of the user with the reference value of the human heart rate stored in the storage unit, and send a control signal to the alarm module if the heart rate of the user is not equal to the reference value of the human heart rate, and the alarm module controls the infrared touch screen to display alarm information when the control signal is received.

3. The portable electronic device as described in claim 1, further comprising a storage unit storing a reference value range of a human heart rate, wherein the processor further comprises an alarm module, the data processing module is further configured to compare the heart rate data of the user with the reference value range of the human heart rate stored in the storage unit, and send a control signal to the alarm module if the heart rate of the user does not fall in the reference value range, and the alarm module controls the infrared touch screen to display alarm information when the control signal is received.

4. The portable electronic device as described in claim 1, wherein the infrared touch screen comprises a monitoring region and a result displaying region, the monitoring region is configured for allowing the user to place the body part thereon for heart rate detection, and the result displaying region is configured for displaying the heart rate monitoring result.

5. The portable electronic device as described in claim 4, wherein the heart rate monitoring result is selected from the group consisting of a heart rate figure, and information of heart beat or pulse.

6. The portable electronic device as described in claim 5, wherein the information of heart beat or pulse is a number of heart beats per minute of the user.

7. The portable electronic device as described in claim 1, being a mobile terminal, electronic reader, a tablet computer, or a digital phone frame.
